(19) United States
(12) Patent Application Publication
(10) Pub. No.: US 2006/0211588 A1
(43) Pub. Date: Sep. 21, 2006

(54) Composition containing a urea compound and a polymer, uses thereof

(75) Inventors: Angeles Fonolla Moreno, Paris (FR); Bertrand Piot, Paris (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/355,164

(22) Filed: Feb. 16, 2006

Related U.S. Application Data
(60) Provisional application No. 60/672,865, filed on Apr. 20, 2005.

(30) Foreign Application Priority Data
Mar. 17, 2005 (FR) 05 50683

(51) Int. Cl. A61K 8/65

(52) U.S. Cl. 510/126

(57) ABSTRACT

The invention relates to a cosmetic cleansing composition containing, in an aqueous medium, (1) at least one nonionic surfactant, (2) at least one hydroxylated urea compound, and (3) at least one polymeric quaternary ammonium salt. The invention also relates to the use of the composition as a cleansing and/or makeup-removing product for the skin and/or the lips, or as a hair cleansing product.

### REFERENCE TO PRIOR APPLICATIONS

This application claims priority to U.S. provisional application 60/672,865 filed Apr. 24, 2005, and to French patent application 0550683 filed Mar. 17, 2005, both incorporated herein by reference.

### FIELD OF THE INVENTION

The invention relates to a composition comprising, in an aqueous medium, a hydroxylated urea compound, a nonionic foaming surfactant, and a polymeric quaternary ammonium salt, and to its uses for cleansing and/or removing makeup from human skin, the lips and/or the hair. In this regard, the composition of the invention is preferably a cosmetic cleansing composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cleansing of the skin is very important for facial care. It should be as efficient as possible since greasy residues such as excess sebum, the residues of the cosmetic products used daily and makeup products accumulate in the folds of the skin and can block the skin pores and result in the appearance of spots. One means for satisfactorily cleansing the skin is to use foaming cleansing products.

One of the major difficulties in the formulation of foaming products consists in developing products that have very good foaming power to ensure good cleansing, and that can be rinsed off easily, while at the same time respecting the skin barrier, i.e. without drying out the skin.

For the formulation of a product of such quality, it is necessary to use a high content of foaming surfactants to ensure a rapid, abundant, dense foam, which is easy to spread and which will be able to be rinsed off easily, and to add a moisturizer, which proves to be essential in order to have good tolerance and to ensure respect of the hydrolipid protective film and thus to avoid drying-out of the skin.

Now, it has already been found that most of the compositions that have good foaming properties contain surfactants that excessively dry out the skin, and that the addition of glycerol, which is a conventionally known moisturizing active agent, makes it possible to attenuate this drying-out effect but has the drawback of reducing the foaming power of the composition (poor initiation of foaming) and of making the composition more difficult to rinse off. In addition, in order to have a moisturizing effect, it is necessary to have a certain amount of glycerol, and the foaming composition obtained has a heavy and tacky feel when applied to the skin.

There is thus still a need for cosmetic cleansing compositions that do not dry out the skin and are therefore moisturizing, while nevertheless having good foaming power (good initiation of foaming) and good rinseability, and also a light, non-tacky feel when applied to the skin.

### SUMMARY OF THE INVENTION

The inventors have discovered, surprisingly, that compositions containing a combination of nonionic foaming surfactant, polymeric quaternary ammonium salt, and hydroxylated urea compound solve the problems identified above, this composition forming the basis of the present invention.

### DETAILED DESCRIPTION OF THE PREFERRED EMBODIMENTS

Thus, one subject of the invention is a composition preferable used for cosmetic cleansing comprising, in an aqueous medium, (1) at least one nonionic foaming surfactant, (2) at least one compound of formula (I) below: 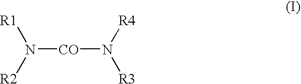
in which:
R1, R2, R3 and R4 represent, independently of each other, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts, solvates and isomers thereof, and (3) at least one polymeric quaternary ammonium salt.

Admittedly, it is known practice from document DE 2 703 185 to introduce urea derivatives of formula (I) into cleansing compositions, but there was nothing to suggest that combining this derivative with nonionic surfactants and a quaternary ammonium salt would produce a composition not only having good moisturizing power, but also having a light, non-tacky feel and moreover having good initiation of foaming, even in the presence of a large amount of urea compound.

Thus, the composition of the invention has the advantage of not drying out the skin, while at the same time having good foaming power and good rinseability. The foam obtained is abundant, dense and creamy, and rinses off very well.

The term “aqueous medium” means herein a medium containing water and optionally one or more water-soluble organic solvents, etc. In this aqueous medium, the amount of water is preferably at least 20% by weight; it preferably ranges from 20% to 95% or 100% by weight, better still from 30% to 90% by weight and even better still from 40% to 85% by weight relative to the total weight of the composition.

In addition, since the composition of the invention is preferably a cosmetic composition, it typically contains a physiologically acceptable medium. The term “physiologically acceptable medium” means herein a medium that is compatible with the skin, mucous membranes and the eyes. It is preferably a cosmetically acceptable medium, i.e. a medium that also has a pleasant colour, odour and feel and that does not cause any unacceptable discomfort (stinging, tautness or redness) liable to put the consumer off using this composition.

Advantageously, the pH of the aqueous medium is compatible with keratin materials and especially with the skin. This pH preferably ranges from 3 to 8.5, better still from 3.5 to 8 and preferentially from 5 to 7.5.

The composition of the invention is preferably a foaming composition that is rinsed off after application to the skin.

Hydroxylated Urea Compound

The urea compound present in the composition according to the invention is a compound of formula (I) below: 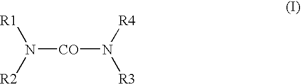
in which:
R1, R2, R3 and R4 represent, independently of each other, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts, solvates and isomers thereof. Of course, mixtures may be used.

In the compounds of formula (I):

preferably, R1 denotes a C2-C6 hydroxyalkyl group and R2, R3 and R4 denote, independently of each other, a hydrogen atom or a C1-C4 alkyl group;

preferentially, R1 denotes a C2-C6 hydroxyalkyl group comprising from 1 to 5 hydroxyl groups and especially one hydroxyl group, and R2, R3 and R4 denote a hydrogen atom;

more preferentially, R1 denotes a C2-C4 hydroxyalkyl group comprising one hydroxyl group and R2, R3 and R4 denote a hydrogen atom.

C1-C4 alkyl groups that may especially be mentioned include methyl, ethyl, n-propyl, isopropyl, n-butyl, isobutyl and tert-butyl groups.

Among the C1-C6 hydroxyalkyl groups that are preferred are those containing only one hydroxyl group and in particular hydroxyethyl, hydroxypropyl, hydroxybutyl, hydroxypentyl and hydroxyhexyl groups.

Salts of such compounds that may be mentioned include the salts of mineral acids, such as sulfuric acid, hydrochloric acid, hydrobromic acid, hydriodic acid, phosphoric acid and boric acid. Mention may also be made of the salts of organic acids, which may comprise one or more carboxylic acid, sulfonic acid or phosphonic acid groups. They may be linear, branched or cyclic aliphatic acids or alternatively aromatic acids. These acids may also comprise one or more hetero atoms chosen from O and N, for example in the form of hydroxyl groups. Mention may especially be made of propionic acid, acetic acid, terephthalic acid, citric acid and tartaric acid.

The term “solvate” means a stoichiometric mixture of the said compound of formula (I) with one or more water molecules or organic solvent molecules, such a mixture being derived from the synthesis of the compound of formula (I).

Preferred compounds of formula (I) that may be mentioned include N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxy-hexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)-methyl]urea; N-ethyl-N′-(2-hydroxyethyl)urea; N,N-bis(2-hydroxyethyl)urea; N,N′-bis(2-hydroxyethyl)-urea; N,N-bis(2-hydroxypropyl)urea; N,N′-bis(2-hydroxy-propyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)-urea; N-(1,3-dihydroxy-2-propyl)-N′-(2-hydroxyethyl)-urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)urea; and mixtures thereof.

Preferably, the compound of formula (I) is N-(2-hydroxyethyl)urea.

The compounds of formula (I) are known compounds and are described especially in patent application DE-A-2 703 185. Among these, N-(2-hydroxyethyl)urea is also commercially available, in the form of a mixture at 50% by weight in water, from the company National Starch under the trade name Hydrovance®.

The compound of formula (I) may be present in the composition according to the invention in any effective amount, such as an amount (of active material) ranging from 0.5% to 30% by weight, preferably ranging from 1% to 20% by weight and preferentially ranging from 2% to 10% by weight relative to the total weight of the composition.

Nonionic Foaming Surfactant

The composition if the invention comprises one or more nonionic foaming surfactants. These surfactants are foaming surfactants capable of cleansing the skin.

Foaming surfactants are detergents and differ from emulsifiers by their HLB value (hydrophilic-lipophilic balance), the HLB being the ratio between the hydrophilic part and the lipophilic part of the molecule. The term HLB is well known to those skilled in the art and is described, for example, in “The HLB system. A time-saving guide to Emulsifier Selection” (published by ICI Americas Inc.; 1984). For emulsifiers, the HLB generally ranges from 3 to 8 for the preparation of W/O emulsions and from 8 to 18 for the preparation of O/W emulsions, whereas foaming surfactants generally have an HLB of greater than 20.

The nonionic foaming surfactants of the composition of the invention may be chosen especially from alkylpolyglucosides (APGs), oxyalkylenated glycerol esters and oxyalkylenated sugar esters, and mixtures thereof. They are preferably APGs.

Alkylpolyglucosides that are preferably used are those containing an alkyl group containing from 6 to 30 carbon atoms and preferably from 8 to 16 carbon atoms, and containing a glucoside group preferably comprising from 1.2 to 3 glucoside units. The alkyl-polyglucosides may be chosen, for example, from decylglucoside (Alkyl-C9/C11-polyglucoside (1.4)), such as the product sold under the name Mydol 10® by the company Kao Chemicals or the product sold under the name Plantacare 2000 UP® by the company Cognis; caprylyl/capryl glucoside, for instance the product sold under the name Plantacare KE3711® by the company Cognis; laurylglucoside, for instance the product sold under the name Plantacare 1200 UP® by the company Cognis; cocoglucoside, for instance the product sold under the name Plantacare 818 UP® by the company Cognis; caprylylglucoside, for instance the product sold under the name Plantacare 810 UP® by the company Cognis; and mixtures thereof.

The oxyalkylenated glycerol esters are especially polyoxyethylenated derivatives of glyceryl esters of fatty acids and hydrogenated derivatives thereof. These oxyalkylenated glycerol esters may be chosen, for example, from hydrogenated and oxyethylenated glyceryl esters of fatty acids such as PEG-200 hydrogenated glyceryl palmate sold under the name Rewoderm LI-S 80 by the company Goldschmidt; oxyethylenated glyceryl cocoates, for instance PEG-7 glyceryl cocoate sold under the name Tegosoft GC by the company Goldschmidt, and PEG-30 glyceryl cocoate sold under the name Rewoderm LI-63 by the company Goldschmidt; and mixtures thereof.

The oxyalkylenated sugar esters are especially polyethylene glycol ethers of fatty acid esters of sugars. These oxyalkylenated sugar esters may be chosen, for example, from oxyethylenated glucose esters such as PEG-120 methyl glucose dioleate sold under the name Glucamate DOE 120 by the company Amerchol.

According to one preferred embodiment of the invention, the nonionic surfactant is an alkylpolyglucoside that may be chosen especially from decylglucoside, caprylyl/capryl glucoside, laurylglucoside, cocoglucoside and caprylylglucoside, and mixtures thereof.

The composition according to the invention may contain any effective amount of nonionic foaming surfactant(s) (as active material) such as, for example, from 1% to 50% by weight, preferably from 1% to 40% by weight and better still from 2% to 30% by weight relative to the total weight of the composition.

The composition according to the invention may contain, besides the one or more nonionic foaming surfactants, one or more foaming surfactants chosen from anionic, amphoteric and zwitterionic surfactants.

Anionic Surfactants

The anionic foaming surfactants that may be added to the composition according to the invention include anionic derivatives of proteins of plant origin or of silk proteins, phosphates and alkyl phosphates, carboxylates, sulfosuccinates, amino acid derivatives, alkyl sulfates, alkyl ether sulfates, sulfonates, isethionates, taurates, alkyl sulfoacetates, polypeptides and anionic derivatives of alkyl-polyglucosides, and mixtures thereof.

The anionic derivatives of proteins of plant origin are protein hydrolysates containing a hydrophobic group, the said hydrophobic group possibly being naturally present in the protein or being added by reacting the protein and/or the protein hydrolysate with a hydrophobic compound. The proteins are of plant origin or derived from silk, and the hydrophobic group may especially be a fatty chain, for example an alkyl chain containing from 10 to 22 carbon atoms. As anionic derivatives of proteins of plant origin, mention may be made more particularly of protein hydrolysates of apple, wheat, soybean or oat, comprising an alkyl chain containing from 10 to 22 carbon atoms, and salts thereof. The alkyl chain can be in particular a lauryl chain and the salt can be a sodium, potassium and/or ammonium salt.

Thus, as protein hydrolysates containing a hydrophobic group, mention may be made, for example, of salts of hydrolysates of silk protein modified with lauric acid, such as the product sold under the name Kawa Silk by the company Kawaken; salts of hydrolysates of wheat protein modified with lauric acid, such as the potassium salt sold under the name Aminofoam W OR by the company Croda (CTFA name: potassium lauroyl wheat amino acids) and the sodium salt sold under the name Proteol LW 30 by the company SEPPIC (CTFA name: sodium lauroyl wheat amino acids); salts of hydrolysates of oat protein comprising an alkyl chain containing from 10 to 22 carbon atoms and more especially salts of hydrolysates of oat protein modified with lauric acid, such as the sodium salt sold under the name Proteol Oat (aqueous 30% solution) by the company SEPPIC (CTFA name: sodium lauroyl oat amino acids); salts of hydrolysates of apple protein, comprising an alkyl chain containing from 10 to 22 carbon atoms, such as the sodium salt sold under the name Proteol APL (30% water-glycol solution) by the company SEPPIC (CTFA name: sodium cocoyl apple amino acids). Mention may also be made of the mixture of lauroyl amino acids (aspartic acid, glutamic acid, glycine and alanine)

neutralized with sodium N-methylglycinate, sold under the name Proteol SAV 50 S by the company SEPPIC (CTFA name: sodium cocoyl amino acids).

Phosphates and alkyl phosphates that may be mentioned, for example, include monoalkyl phosphates and dialkyl phosphates, such as the lauryl monophosphate sold under the name MAP 20® by the company Kao Chemicals, the potassium salt of dodecylphosphoric acid, as a mixture of monoester and diester (mainly diester), sold under the name Crafol AP-31® by the company Cognis, the mixture of monoester and diester of octylphosphoric acid, sold under the name Crafol AP-20® by the company Cognis, the mixture of ethoxylated (7 mol of EO) phosphoric acid monoester and diester of 2-butyloctanol, sold under the name Isofol 12 7 EO-Phosphate Ester® by the company Condea, the potassium salt or triethanolamine salt of monoalkyl (C12-C13) phosphate sold under the references Arlatone MAP 230K-40® and Arlatone MAP 230T-60® by the company Uniqema, and the potassium lauryl phosphate sold under the name Dermalcare MAP XC-99/09® by the company Rhodia Chimie, and the potassium cetyl phosphate sold under the name Arlatone MAP 160K by the company Uniqema.

Carboxylates that may be mentioned include:
amido ether carboxylates (AEC), for instance sodium laurylamido ether carboxylate (3 EO) sold under the name Akypo Foam 30° by the company Kao Chemicals;
polyoxyethylenated carboxylic acid salts, for instance oxyethylenated (6 EO) sodium lauryl ether carboxylate (65/25/10 C₁₂₋₁₄₋₁₆) sold under the name Akypo Soft 45 NV® by the company Kao Chemicals; polyoxyethylenated and carboxymethylated fatty acids of olive oil origin sold under the name Olivem 400® by the company Biologia E Technologia; oxyethylenated (6 EO) sodium tridecyl ether carboxylate sold under the name Nikkol ECTD-6NEX® by the company Nikkol;
fatty acid salts (soaps) containing a C6 to C22 alkyl chain, neutralized with an organic or mineral base such as potassium hydroxide, sodium hydroxide, triethanolamine, N-methylglucamine, lysine or arginine.

Amino acid derivatives that may especially be mentioned include alkaline salts of amino acids, such as:
sarcosinates, for instance the sodium lauroyl sarcosinate sold under the name Sarkosyl NL 97® by the company Ciba or sold under the name Oramix L 30® by the company SEPPIC, the sodium myristoyl sarcosinate sold under the name Nikkol Sarcosinate MN® by the company Nikkol or the sodium palmitoyl sarcosinate sold under the name Nikkol Sarcosinate PN® by the company Nikkol;
alaninates, for instance the sodium N-lauroyl-N-methylamidopropionate sold under the name Sodium Nikkol Alaninate LN 30® by the company. Nikkol or sold under the name Alanone ALE® by the company Kawaken, and the triethanolamine N-lauroyl-N-methylalanine sold under the name Alanone Alta® by the company Kawaken;
glutamates, for instance the triethanolamine monococoylglutamate sold under the name Acylglutamate CT-12® by the company Ajinomoto and the triethanolamine lauroylglutamate sold under the name Acylglutamate LT-12® by the company Ajinomoto;
aspartates, for instance the mixture of triethanolamine N-lauroyl aspartate and of triethanolamine N-myristoyl aspartate, sold under the name Asparack® by the company Mitsubishi;
glycine derivatives (glycinates), for instance the sodium N-cocoylglycinate sold under the names Amilite GCS-12° and Amilite GCK 12 by the company Ajinomoto;
citrates, such as the oxyethylenated (9 mol) citric monoester of cocoyl alcohols sold under the name Witconol EC 1129 by the company Goldschmidt;
galacturonates, such as the sodium dodecyl-D-galactoside uronate sold by the company Soliance.

Examples of sulfosuccinates that may be mentioned include the oxyethylenated (3 EO) lauryl monosulfosuccinate (70/30 C12/C14) sold under the names Setacin 103 Special® and Rewopol SB-FA 30 K 4® by the company witco, the disodium salt of a C12-C14 alkyl hemisulfosuccinate, sold under the name Setacin F Special Paste® by the company Zschimmer Schwarz, the oxyethylenated (2 EO) disodium oleamidosulfosuccinate sold under the name Standapol SH 135® by the company Cognis, the oxyethylenated (5 EO) laurylamide monosulfosuccinate sold under the name Lebon A-5000® by the company Sanyo, the oxyethylenated (10 EO) disodium salt of lauryl citrate monosulfosuccinate sold under the name Rewopol SB CS 50 by the company Witco, and the ricinoleic monoethanolamide monosulfosuccinate sold under the name Rewoderm S 1333® by the company Witco. Polydimethylsiloxane sulfosuccinates may also be used, such as disodium PEG-12 dimethicone sulfosuccinate sold under the name Mackanate-DC30 by the company Macintyre.

Examples of alkyl sulfates that may be mentioned include triethanolamine lauryl sulfate (CTFA name: TEA-lauryl sulfate) such as the product sold by the company Huntsman under the name Empicol TL40 FL or the product sold by the company Cognis under the name Texapon T42, which are products in the form of an aqueous 40% solution. Mention may also be made of ammonium lauryl sulfate (CTFA name: ammonium lauryl sulfate), such as the product sold by the company Huntsman under the name Empicol AL 30FL, which is an aqueous 30% solution.

Examples of alkyl ether sulfates that may be mentioned include sodium lauryl ether sulfate (CTFA name: sodium laureth sulfate), for instance the product sold under the names Texapon N40 and Texapon AOS 225 UP by the company Cognis, and ammonium lauryl ether sulfate (CTFA name: ammonium laureth sulfate), for instance the product sold under the name Standapol EA-2 by the company Cognis.

Examples of sulfonates that may be mentioned include α-olefin sulfonates, for instance the sodium α-olefin sulfonate (C14-16) sold under the name Bio-Terge AS-40® by the company Stepan, sold under the names Witconate AOS Protege® and Sulframine AOS PH 12® by the company Witco or sold under the name Bio-Terge AS-40 CG® by the company Stepan, the sodium secondary olefin sulfonate sold under the name Hostapur SAS 30® by the company Clariant; linear alkyl aryl sulfonates, for instance the sodium xylenesulfonate sold under the names Manrosol SXS30®, Manrosol SXS40® and Manrosol SXS93® by the company Manro.

Isethionates that may be mentioned include acylisethionates, for instance sodium cocoylisethionate, such as the product sold under the name Jordapon CI P® by the company Jordan.

Taurates that may be mentioned include the sodium salt of palm kernel oil methyltaurate sold under the name Hostapon CT Pate® by the company Clariant; N-acyl N-methyltaurates, for instance the sodium N-cocoyl N-methyltaurate sold under the name Hostapon LT-SF® by the company Clariant or sold under the name Nikkol CMT-30-T® by the company Nikkol, and the sodium palmitoyl methyltaurate sold under the name Nikkol PMT® by the company Nikkol.

The anionic derivatives of alkylpolyglucosides may be, in particular, glyceryl ethers, carbonates, sulfosuccinates, tartrates and citrates obtained from alkylpolyglucosides. Mention may be made, for example, of the sodium salt of cocoylpolyglucoside (1,4) tartaric ester, sold under the name Eucarol AGE-ET® by the company Cesalpinia, the disodium salt of cocoylpolyglucoside (1,4)sulfosuccinic ester, sold under the name Essai 512 MP® by the company SEPPIC, and the sodium salt of cocoylpolyglucoside (1,4) citric ester, sold under the name Eucarol AGE-EC® by the company Cesalpinia.

Amphoteric and Zwitterionic Foaming Surfactants

Useful amphoteric and zwitterionic surfactants include those chosen, for example, from betaines, N-alkylamidobetaines and derivatives thereof, sultaines, alkyl polyaminocarboxylates and alkylamphoacetates, and mixtures thereof.

Betaines that may be mentioned especially are alkylbetaines, for example cocobetaine, for instance the product sold under the name Dehyton AB-30® by the company Cognis, laurylbetaine, for instance the product sold under the name Genagen KB® by the company Clariant, oxyethylenated (10 EO) laurylbetaine, for instance the product sold under the name Lauryl Ether (10 EO) Betaine® by the company Shin Nihon Rica, and oxyethylenated (10 EO) stearylbetaine, for instance the product sold under the name Stearyl Ether (10 EO) Betaine® by the company Shin Nihon Rica.

Among the N-alkylamidobetaines and derivatives thereof that may be mentioned, for example, are the cocamidopropylbetaine sold under the name Lebon 2000 HG® by the company Sanyo, or sold under the name Empigen BB® by the company Albright & Wilson, and the lauramidopropyl betaine sold under the name Rewoteric AMB12P® by the company Witco.

Sultaines that may be mentioned include hydroxylsultaines, such as cocoylamidopropylhydroxysultaine, for instance the product sold under the name Rewoteric AM CAS by the company Goldschmidt-Degussa, or the product sold under the name Crosultaine C-50® by the company Croda.

Alkyl polyaminocarboxylates (APACs) that may be mentioned include the sodium cocoylpolyaminocarboxylate sold under the name Ampholak 7 CX/C® and Ampholak 7 CX® by the company Akzo Nobel, the sodium stearylpolyamidocarboxylate sold under the name Ampholak 7 TX/C by the company Akzo Nobel and the sodium carboxymethyloleylpolypropylamine sold under the name Ampholak XO7/C® by the company Akzo Nobel.

Alkylamphoacetates that may be mentioned, for example, include N-disodium N-cocoyl-N-carboxymethoxyethyl-N-carboxymethylethylenediamine (CTFA name: disodium cocoamphodiacetate), for instance the product sold under the name Miranol C2M Concentré NP® by the company Rhodia, and N-sodium N-cocoyl-N-hydroxyethyl-N-carboxymethylethylenediamine (CTFA name: sodium cocamphoacetate), and sodium cocoamphohydroxypropyl sulfonate sold under the name Miranol CSE by the company Rhodia.

The anionic, amphoteric and zwitterionic foaming surfactants, when they are present in the composition of the invention, may be in an amount (of active material) ranging, for example, from 0.5% to 15% by weight and preferably from 1% to 10% by weight relative to the total weight of the composition.

According to one preferred embodiment of the invention, the composition contains as foaming surfactant, other than the nonionic surfactant, at least one anionic derivative of proteins of plant origin or of silk proteins.

According to another preferred embodiment of the invention, the composition of the invention contains at least one alkylpolyglucoside and at least one anionic derivative of proteins of plant origin or of silk proteins. The composition according to the invention containing the hydroxylated urea derivative, the claimed polymer and this combination of surfactants has the advantage of being very mild and of being well tolerated, while at the same time having satisfactory moisturizing and foaming properties.

Polymeric Quaternary Ammonium Salt

The polymeric quaternary ammonium salts are cationic or amphoteric polymers containing at least one quaternized nitrogen atom. Polymeric quaternary ammonium salts that may especially be mentioned include the Polyquaternium products (CTFA name), which give the foaming cream-softness and creaminess. These polymers may preferably be chosen from the following polymers:
Polyquaternium-5, such as the product Merquat 5 sold by the company Calgon;
Polyquaternium-6, such as the product Salcare SC 30 sold by the company Ciba, and the product Merquat 100 sold by the company Calgon;
Polyquaternium-7, such as the products Merquat S, Merquat 2200 and Merquat 550 sold by the company Calgon; and the product Salcare SC 10 sold by the company Ciba;
Polyquaternium-10, such as the product Polymer JR400 sold by the company Amerchol;
Polyquaternium-11, such as the products Gafquat 755, Gafquat 755N and Gafquat 734 sold by the company ISP;
Polyquaternium-15, such as the product Rohagit KF 720 F sold by the company Rohm;
Polyquaternium-16, such as the products Luviquat FC905, Luviquat FC370, Luviquat HM552 and Luviquat FC550 sold by the company BASF;
Polyquaternium-22, such as the product Merquat 280 sold by the company Calgon;
Polyquaternium-28, such as the product Styleze CC10 sold by the company ISP;
Polyquaternium-39, such as the product Merquat Plus 3330 sold by the company Calgon;
Polyquaternium-44, such as the product Luviquat Care sold by the company BASF;
Polyquaternium-46, such as the product Luviquat

Hold sold by the company BASF;
Polyquaternium-47, such as the product Merquat 2001 sold by the company Calgon.

Preferably, the quaternary ammonium salts are chosen from Polyquaternium-7, Polyquaternium-10, Polyquaternium-39 and Polyquaternium-47, and mixtures thereof.

The polymeric quaternary ammonium salts may be present in any effective amount (of active material), for example, from 0.05% to 10% by weight and better still from 0.1% to 5% by weight relative to the total weight of the composition.

Adjuvants

The aqueous medium of the composition according to the invention may contain, besides water, one or more water-soluble solvents chosen from lower alcohols (monoalcohols) containing from 1 to 6 carbon atoms, such as ethanol; and polyols such as glycerol, glycols, for instance butylene glycol, isoprene glycol, propylene glycol or polyethylene glycols such as PEG-8; sorbitol; sugars such as glucose, fructose, maltose, lactose or sucrose; and mixtures thereof, provided that these compounds do not impair the desired properties of the composition according to the invention. The amount of solvent(s) in the composition of the invention may range, for example, from 0.1% to 10% by weight, preferably from 0.5% to 5% by weight, better still from 0.5% to 5% by weight and even better still from 0.5% to 3% by weight relative to the total weight of the composition.

The compositions of the invention may also contain adjuvants. Adjuvants that may be mentioned, for example, include fragrances, preserving agents, sequestering agents (EDTA), pigments, fillers, especially exfoliant fillers, soluble dyes, sunscreens, cosmetic or dermatological active agents such as moisturizers, for instance hyaluronic acid, ceramides, water-soluble or liposoluble vitamins, for instance vitamin C and derivatives thereof such as vitamin CG, antiseptics, antiseborrhoeic agents, antimicrobial agents such as benzoyl peroxide, salicylic acid, triclosan and azelaic acid, and also optical brighteners. The amounts of these various adjuvants are those conventionally used in the field under consideration, for example from 0.01% to 5% of the total weight of the composition. These adjuvants and their concentrations should be such that they do not modify the desired property for the composition of the invention.

Examples of exfoliants that may be mentioned include exfoliant or scrubbing particles of mineral, plant or organic origin. Thus, polyethylene beads or powder, Nylon powder, polyvinyl chloride powder, pumice, ground apricot kernel or walnut shell, sawdust, glass beads or alumina, and mixtures thereof, may be used, for example. These particles may be present in an amount ranging, for example, from 0.5% to 40% by weight, preferably from 1% to 20% by weight and better still from 1% to 10% by weight relative to the total weight of the composition. When the composition contains exfoliant particles, it may especially constitute a scrubbing composition for facial skin or the body.

The compositions of the invention may also contain polymers, especially anionic and nonionic polymers.

Anionic polymers that may especially be mentioned include those comprising at least one hydrophobic chain, and in particular those derived from acrylic or methacrylic acid, for instance the acrylates/steareth-20 methacrylate copolymer sold under the name Aculyn 22 by the company Rohm & Haas (CTFA name: Acrylates/Steareth-30 Methacrylate copolymer); the (meth)acrylic acid/ethyl acrylate/behenyl methacrylate oxyethylenated (25 OE) terpolymer, as an aqueous emulsion sold under the name Aculyn 28 by the company Rohm & Haas; the acrylic acid/monocetyl itaconate oxyethylenated (20 OE) copolymer, as an aqueous 30% dispersion sold under the name Structure 3001 by the company National Starch; the acrylic acid/monostearyl itaconate oxyethylenated (20 OE) copolymer, as an aqueous 30% dispersion sold under the name Structure 2001 by the company National Starch; the copolymer of acrylates/acrylate modified with polyoxyethylenated (25 OE) C₁₂-C₂₄ alcohols, in the form of a latex containing 30-32% copolymer, sold under the name Synthalen W2000 by the company 3V SA, and also the copolymers sold under the names Pemulen or Carbopol by the company Noveon, for instance the acrylate/C₁₀-C₃₀-alkylacrylate copolymer, such as the products Pemulen TR1, Pemulen TR2 or Carbopol 1382 (CTFA name:

Acrylates/C10-30 Alkyl acrylate Crosspolymer).

The composition may also contain polymers comprising at least one monomer containing a sulfonic group, and especially 2-acrylamido-2-methylpropanesulfonic acid (AMPS) polymers and copolymers, such as:

the crosslinked and neutralized 2-acrylamido-2-methylpropanesulfonic acid homopolymer sold by the company Clariant under the trade name “Hostacerin AMPS” (CTFA name: ammonium polyacryldimethyltauramide);

crosslinked anionic copolymers of acrylamide or methacrylamide and of 2-acrylamido-2-methylpropanesulfonic acid, especially those in the form of a W/O emulsion, such as those sold under the name Sepigel 305 by the company SEPPIC (CTFA name: Polyacrylamide/C13-14 Isoparaffin/Laureth-7), under the name Simulgel 600 by the company SEPPIC (CTFA name: Acrylamide/Sodium acryloyldimethyltaurate copolymer/Isohexadecane/Poly-sorbate 80);

copolymers of (meth)acrylic acid or of (meth)acrylate and of 2-acrylamido-2-methylpropanesulfonic acid, especially those in the form of a W/O emulsion, such as those sold under the name Simulgel NS by the company SEPPIC (copolymer of sodium acrylamido-2-methylpropanesulfonate/hydroxyethyl acrylate as a 40% inverse emulsion in Polysorbate 60 and squalane) (CTFA name: Hydroxyethyl acrylate/Sodium acryloyldimethyltaurate copolymer/squalane/Polysorbate 60), or those sold under the name Simulgel EG by the company SEPPIC (copolymer of acrylic acid/acrylamido-2-methylpropanesulfonic acid in the form of the sodium salt, as a 45% inverse emulsion in isohexadecane/water) (CTFA name: Sodium acrylate/Sodium acryloyldimethyltaurate copolymer/Isohexadecane/Polysorbate 80);

copolymers of 2-acrylamido-2-methylpropanesulfonic acid and of vinylpyrrolidone or of vinylformamide, such as the products sold under the name Aristoflex AVC by the company Clariant;

hydrophobic-modified AMPS polymers such as, especially, the copolymer of AMPS and of ethoxylated C12-C14 alkyl methacrylate (non-crosslinked copolymer obtained from Genapol LA-070 and from AMPS) (CTFA name: Ammonium Acryloyldimethyltaurate/Laureth-7 Methacrylate Copolymer) sold under the name Aristoflex LNC by the company Clariant, and the copolymer of AMPS and of ethoxylated (25 EO) stearyl methacrylate (copolymer crosslinked with trimethylolpropane triacrylate, obtained from Genapol T-250 and AMPS) (CTFA name: Ammonium Acryloyldimethyltaurate/Steareth-25 Methacrylate Crosspolymer) sold under the name Aristoflex HMS by the company Clariant.

These polymers may be present in any effective amount (of active material), for example, from 0.05% to 10% by weight and better still from 0.1% to 5% by weight relative to the total weight of the composition.

The compositions according to the invention may be used on any keratin material, such as the skin, the scalp, the hair, the eyelashes, the eyebrows, the nails or mucous membranes, especially as a hygiene product, for example as a product for cleansing the skin, mucous membranes and/or the hair, in particular as a product for cleansing and/or removing makeup from the skin (of the face and/or the body), as a shower product (two-in-one product), as a shampoo and hair conditioner, as a shaving product, as a rinse-off mask, and as an exfoliant product (also known as a desquamating or scrubbing product) either for the face or for the body or the hands, after addition of exfoliant particles.

A subject of the invention is also the cosmetic use of the composition as defined above, as a skin cleansing and/or makeup-removing product, as a shower product, as a shampoo, as a hair-conditioning product, as a shaving product, as a rinse-off mask or as an exfoliant product.

Another subject of the invention is a process for cleansing a keratin material, such as the skin, the scalp, the hair, the eyelashes, the eyebrows, the nails or mucous membranes, wherein a composition as defined above is applied to the keratin material, and is rinsed off.

The keratin material is preferably the skin.

The compositions according to the invention may be used, for example, in the following manner:

1) when the compositions according to the invention are mild facial cleansing products, they may be used in the following manner:

the product is worked into a lather in the hands with water,

the lather is applied to the face,

the face is cleansed,

the product is rinsed off with water;

2) when the compositions according to the invention are makeup-removing products, they may be used as indicated above, but they may also be applied dry to the face and then massaged until satisfactory makeup removal has been obtained, and rinsed off with water, or alternatively they may be applied using cotton wool;

3) they may be used as two-in-one shower products, as shampoos and/or hair conditioners, as rinse-off masks or as shaving products, in the usual manner for the use of these products.

The examples that follow serve to illustrate the invention without, however, being limiting in nature. The amounts indicated are weight percentages unless otherwise mentioned, and they correspond, unless otherwise mentioned, to the amount of starting material rather than to the amount of active material. The names of the compounds used are indicated as the CTFA name, the chemical name or the trade name.

### Example 1 According to the Invention

| Foaming composition | Example 1 |
|---|---|
| PHASE I | |
| Water | qs 100% |
| Hydrovance (1) | 14% |
| (i.e. 7% active material) | |
| Preserving agent | 0.3% |
| Disodium EDTA | 0.2% |
| Aculyn 22 (2) | 2.2% |
| Carbopol 1382 | 0.4% |
| Hostacerin AMPS | 0.6% |
| Triethanolamine | 0.7% |
| PHASE II | |
| Decylglucoside (3) | 5% |
| Cocoglucoside (4) | 3% |
| Ammonium lauryl sulfate (5) | 2% |
| TEA-lauryl sulfate (6) | 0.5% |
| Sodium lauryl OAT amino acids (7) | 2% |
| Triethanolamine | 0.01% |
| PHASE III | |
| Polyquaternium-47 | 0.3% |
| Fragrance | 0.1% |

(1) Hydrovance=N-(2-hydroxyethyl)urea as an aqueous solution at 50% by weight
(2) Acrylic copolymer (as an aqueous dispersion containing 30% active material)
(3) Oramix NS 10 (aqueous solution containing 55% active material)
(4) Plantacare 1200 UP (aqueous solution containing 50% active material)
(5) Empicol AL 30/FL (aqueous 30% solution)
(6) Empicol TL 40/FL (aqueous 40% solution)
(7) Proteol OAT (30% solution)
Procedure: phase I was prepared, and the compounds of phase II and then phase III were gradually added thereto.

A composition was obtained which foams well and which gave a foam that starts well and is abundant. In addition, the composition rinsed off well and was well tolerated by the skin. It may be advantageously used for cleansing facial skin.

Comparative Example 1: the hydroxyethylurea in Example 1 was replaced with glycerol. A composition was obtained which gave a foam that starts more slowly, and that is much less abundant and less dense than that of Example 1 with hydroxyethylurea. In addition it rinsed off more slowly.

### Example 2 According to the Invention

| Foaming/exfoliant composition | Example 2 |
|---|---|
| PHASE I | |
| Water | qs 100% |
| Hydrovance (1) | 20% |
| (i.e. 10% active material) | |
| Preserving agent | 0.3% |
| Disodium EDTA | 0.25% |
| Aculyn 22 (2) | 3% |
| Laponite | 1% |
| Triethanolamine | 0.7% |
| PHASE II | |
| Decylglucoside (3) | 7% |
| Lauroylglucoside (4) | 1% |
| Sodium cocoyl apple amino acids (5) | 6% |
| PEG-60 Hydrogenated castor oil | 0.8% |
| Sodium laureth sulfate (6) | 3% |
| PHASE III | |
| Alumina | 4% |
| Polyethylene beads | 3% |
| Pumice | 0.5% |
| PHASE IV | |
| Benzophenone-4 (screening agent) | 0.15% |
| Fragrance | 0.1% |

(1) Hydrovance=N-(2-hydroxyethyl)urea as an aqueous solution at 50% by weight
(2) Acrylic copolymer (as an aqueous dispersion containing 30% active material)
(3) Oramix NS 10 (aqueous solution containing 55% active material)
(4) Plantacare 1200 UP (aqueous solution containing 50% active material)
(5) Proteol APL (aqueous solution containing 30% active material)
(6) Texapon N40 (aqueous solution containing 28% active material)

The procedure was analogous to that of Example 1.

A composition was obtained which gives a foam that starts well and that is abundant. In addition, the composition rinsed off well and left the skin soft. It constitutes a scrubbing composition that is very well tolerated.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in an aqueous medium,
(1) at least one nonionic foaming surfactant,
(2) at least one compound of formula (I): 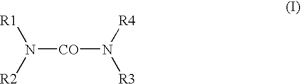
in which:
R1, R2, R3 and R4 represent, independently of each other, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts, solvates and isomers thereof, and
(3) at least one polymeric quaternary ammonium salt.

2. The composition according to claim 1, wherein it comprises at least 20% by weight of water relative to the total weight of the composition.

3. The composition according to claim 1, wherein, in the compounds of formula (I), R1 denotes a C2-C6 hydroxyalkyl group and R2, R3 and R4 denote, independently of each other, a hydrogen atom or a C1-C4 alkyl group.

4. The composition according to claim 1, wherein, in the compounds of formula (I), R1 denotes a C2-C6 hydroxyalkyl group comprising from 1 to 5 hydroxyl groups, and R2, R3 and R4 denote a hydrogen atom.

5. The composition according to claim 4, wherein R1 denotes a C2-C6 hydroxyalkyl group comprising one hydroxyl group.

6. The composition according to claim 1, wherein, in the compounds of formula (I), R1 denotes a C2-C4 hydroxyalkyl group comprising one hydroxyl group and R2, R3 and R4 denote a hydrogen atom.

7. The composition according to claim 1, comprising at least one compound of formula (I) selected from the group consisting of N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)methyl]urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)urea; and mixtures thereof.

8. The composition according to claim 1, comprising N-(2-hydroxyethyl)urea.

9. The composition according to claim 1, wherein the salts of the compounds of formula (I) are chosen from the salts of sulfuric acid, hydrochloric acid, hydrobromic acid, hydriodic acid, phosphoric acid, boric acid, propionic acid, acetic acid, terephthalic acid, citric acid and tartaric acid.

10. The composition according to claim 1, wherein the compound of formula (I) is present in an amount of 0.5% to 30% by weight relative to the total weight of the composition.

11. The composition according to claim 1, wherein the nonionic foaming surfactant is chosen from alkylpolyglucosides, oxyalkylenated glycerol esters, oxyalkylenated sugar esters, and mixtures thereof.

12. The composition according to claim 1, wherein the nonionic foaming surfactant is chosen from decylglucoside, caprylyl/capryl glucoside, laurylglucoside, cocoglucoside and caprylylglucoside, and mixtures thereof.

13. The composition according to claim 1, wherein the amount of nonionic foaming surfactant(s) is 1% to 50% by weight relative to the total weight of the composition.

14. The composition according to claim 1, further comprising one or more foaming surfactants chosen from anionic, amphoteric and zwitterionic surfactants.

15. The composition according to claim 14, wherein the amount of anionic, amphoteric and zwitterionic foaming surfactants is 0.5% to 15% by weight relative to the total weight of the composition.

16. The composition according to claim 1, further comprising an anionic derivative of proteins of plant origin or of silk protein.

17. The composition according to claim 1, wherein the polymeric quaternary ammonium salt is chosen from Polyquaternium products.

18. The composition according to claim 17, wherein the Polyquaternium is chosen from Polyquaternium-7, Polyquaternium-10, Polyquaternium-39 and Polyquaternium-47, and mixtures thereof.

19. The composition according to claim 1, wherein the amount of polymeric quaternary ammonium salt(s) is 0.05% to 10% by weight relative to the total weight of the composition.

20. The composition according to claim 1, wherein it is a skin cleansing and/or makeup-removing product, a shower product, a shampoo, a hair conditioner, a shaving product, a rinse-off mask or an exfoliant product.

21. A process for cleansing a keratin material, wherein a composition according to claim 1 is applied to the keratin material, and is rinsed off.
